(19) United States
(12) Patent Application Publication
(10) Pub. No.: US 2004/0220121 A1
(43) Pub. Date: Nov. 4, 2004

(54) Methods for drug delivery

(76) Inventor: Jasbir Sandhu, Burlington (CA)

Correspondence Address: Michael A. Slavin, Esq., McHale & Slavin, P.A., Suite 402, 4440 PGA Boulevard, Palm Beach Gardens, FL, 33410, US

(21) Appl. No.: 10/429,655

(22) Filed: May 2, 2003

(51) Int. Cl. A61K 31/704; A61K 31/407

(52) U.S. Cl. 514/34; 514/8; 514/410

(57) ABSTRACT

The invention provides methods useful for the delivery of anti-tumor agents to a host having a tumor. The methods of the invention involve the administration of compositions containing a ligand such as human epidermal growth factor (EGF) or human vascular endothelial growth factor (VEGF), an anti-tumor agent and a human transferrin ligand to a host having a tumor.

### CROSS REFERENCE TO RELATED APPLICATIONS

The instant application is related to application Ser. No. 10/______; 10/______; 10/______; 10/______; 10/______; 10/______ ; 10/______; 10/______; 10/______ ; 10/______ and 10/______; all filed on even date herewith under Express Mail labels EV 140261687 US; EV 140261673 US; EV 140261660 US; EV 140261585 US; EV 140261571 US; EV 140261568 US; EV 140261554 US; EV 140261537 US; EV 140261523 US; EV 001630864 US and EV 001630847 US; the contents of which are each herein incorporated by reference.

### FIELD OF THE INVENTION

The instant invention relates generally to methods useful for the delivery of anti-tumor agents to a host having a tumor; particularly to methods useful/for selective delivery of anti-tumor agents to a host having a tumor and most particularly to methods useful for the selective delivery of anti-tumor agents involving the administration of compositions to hosts having tumors wherein said compositions contain a ligand such as human epidermal growth factor (EGF) or human vascular endothelial growth factor (VEGF) and at least one anti-tumor agent operatively linked to a human transferrin ligand.

### BACKGROUND OF THE INVENTION

Malignant disease is a major cause of mortality and morbidity in most countries. Treatment with anti-tumor agents is a therapeutic option of increasing importance, especially for systemic, metastatic disease which has progressed passed the point of surgical curability. Malignant tumors are heterogeneous with regard to their genetics, biology and biochemistry and often possess innate or treatment-induced resistance to therapy. These properties all work against the ease of development of efficient curative treatment. Thus, progress in the development of agents that can cure human cancer has been extremely slow.

As used herein, an anti-tumor agent is defined as any substance that is capable of inhibiting the proliferation of or killing cells of tumor tissues. There are several broad categories of anti-tumor agents, including, alkylating agents, anti-tumor antibiotics, plant alkaloids, anti-metabolites and hormonal agonists and antagonists(see U.S. Pat. No. 6,495,553, issued to Shepard, for a background discussion of anti-tumor agents).

Alkylating agents are very reactive compounds which have the ability to either substitute alkyl groups for hydrogen atoms or to cause methylation and chloroethylation of DNA and proteins. Alkylation of nucleic acids is a critical cytotoxic action as it interferes with DNA replication and RNA transcription. Illustrative, albeit non-limiting examples of alkylating agents are mechlorethamine, chlorambucil, melphalan, cyclophosphamide, ifosfamide, thiotepa, busulfan, dacarbazine, carnustine, lomustine, cisplatin, carboplatin, procarbazine and altretamine.

Anti-tumor antibiotics are natural products of the soil fungus, Streptomyces. These antibiotics are capable of binding DNA, usually through intercalation, to result in the unwinding of the DNA helix. The unwinding impairs the ability of DNA to function as a template for nucleic acid synthesis. These antibiotics are also capable of forming damaging free radicals and chelating metal ions. Additionally, anti-tumor antibiotics may inhibit topoisomerase II, an enzyme important for cell division. Illustrative, albeit non-limiting examples of anti-tumor antibiotics are doxorubicin, daunorubicin, idarubicin, mitoxantrone bleomycin, dactinomycin, carminomycin, detorubicin, epirubicin, esorubicin, adriamycin, mitomycin C, plicamycin and streptozocin.

Plants have also provided useful anti-tumor agents. Vinca alkaloids, such as vincristine and vinblastine, are capable of binding microtubular proteins of dividing cells. This binding alters the structure of tubulin addition and loss at the ends of mitotic spindles, ultimately resulting in mitotic arrest and cell death. Similar microtubular proteins are found in nervous tissue, thus vinca alkaloids are also neurotoxic. Paclitaxel (taxol) is another plant-derived agent useful for interfering with microtubular protein function. Epipodophyllotoxins, such as etoposide and teniposide, are capable of inhibiting topoisomerase II, an enzyme important for cell division.

Anti-metabolites are structural analogs of normal metabolites that are required for cell function and replication. Anti-metabolites function by interacting with cellular enzymes. Illustrative, albeit non-limiting examples of anti-metabolites are methotrexate, 5-fluorouracil (5-FU), floxuridine (FUDR), cytarabine, 6-mercaptopurine (6-MP), 6-thioguanine, deoxycoformycin, fludarabine, 2-chlorodeoxyadenosine, and hydroxyurea.

Many types of cancer are affected by hormonal changes, thus, endocrine manipulation is an effective therapy for several forms of neoplastic disease. A wide variety of hormones and hormone antagonists have been developed for potential use in cancer treatment. Illustrative, albeit non-limiting examples of hormonal agents are diethylstilbestrol, tumoxifen, megestrol acetate, dexamethasone, prednisone, aminoglutethimide, leuprolide, goserelin, flutamide, and octreotide acetate.

The major problem currently associated with the use of anti-tumor agents is low selectivity of the anti-tumor agents. In recent years there has been an increasing awareness that the lack of selectivity of anti-tumor agents is related to their pharmacokinetic properties, meaning, for example, that an anti-tumor agent has a short-half life in the bloodstream with rapid diffusion throughout the body resulting in even distribution of the anti-tumor agent in all tissues. Even distribution of the anti-tumor agent results with insufficient concentration of the anti-tumor agent at the site of the tumor for tumor destruction but results with sufficient concentration in non-diseased tissues to produce severe toxic side effects. Ideally, an anti-tumor agent should be present in an appropriate concentration at the site of the tumor in vivo and in a reduced concentration in other tissues.

The majority of anti-tumor agents that are now used in cancer therapy act by an anti-proliferative mechanism. However, if a tumor does not have a high proportion of cells that are rapidly proliferating, it is not particularly sensitive to this type of agent. Moreover, most anti-tumor agents have steep dose-response curves. Because of host toxicity, treatment has to be discontinued at dose levels that are well below the dose that would be required to kill all viable tumor cells.

Another side effect associated with cancer therapies is the toxic effect of the anti-tumor agent on the normal host tissues that are the most rapidly dividing, such as the bone marrow, gut mucosa and cells of the lymphoid system. The agents also exert a variety of other adverse effects, including neurotoxicity; negative effects on sexuality and gonadal function; and cardiac, pulmonary, pancreatic and hepatic toxicities; vascular and hypersensitivity reactions, and dermatological reactions.

Hematologic toxicity is the most dangerous form of side effect for many of the anti-tumor agents used in clinical practice. The most common hematologic toxicity is neutropenia, with an attendant high risk of infection. Life-threatening thrombocytopenia and bleeding may also occur. Cancer therapy may also induce qualitative defects in the function of both polymorphonuclear leukocytes and platelets.

Most of the commonly used anti-tumor agents are capable of suppressing both cellular and humoral immunity. Infections commonly lead to the death of patients with advanced cancer, and impaired immunity as a result of treatment with anti-tumor agents may contribute to such deaths. Chronic, delayed immunosuppression may also result from cancer chemotherapy.

Neurotoxicity can result from cancer treatment, such as, arachnoiditis; myelopathy or encephalomyelopathy; chronic encephalopathies and the somnolence syndrome; acute encephalopathies; peripheral neuropathies; and acute cerebellar syndromes or ataxia.

Many of the commonly employed anti-tumor agents are mutagenic as well as teratogenic. Some, including procarbazine and the alkylating agents, are clearly carcinogenic. This carcinogenic potential is primarily seen as delayed acute leukemia in patients treated with polyfunctional alkylating agents and inhibitors of topoisomerase II, such as etoposide and the anthracycline antibiotics. Cancer therapy has also been associated with cases of delayed non-Hodgkin's lymphoma and solid tumors.

The clinical usefulness of an anti-tumor agent may be severely limited by the emergence of malignant cells resistant to that drug. These resistant cells appear after several treatments with the drug. A number of cellular mechanisms are probably involved in drug resistance, e.g., altered metabolism of the drugs, impermeability of the cell to the active compound or accelerated drug elimination from the cell, altered specificity of an inhibited enzyme, increased production of a target molecule, increased repair of cytotoxic lesions, or the bypassing of an inhibited reaction by alternative biochemical pathways. In some cases, resistance to one drug may confer resistance to other, biochemically distinct drugs. Amplification of certain genes is involved in resistance to therapy. Amplification of the gene encoding dihydrofolate reductase is related to resistance to methotrexate, while amplification of the gene encoding thymidylate synthase is related to resistance to treatment with 5-fluoropyrimidines.

Considering that many of the toxic side effects referred to above are the result of low selectivity, it is evident that a need exists for increased selectivity. Attachment of anti-tumor agents to soluble protein ligands that are capable of specifically targeting tumors is one approach to increasing selectivity of anti-tumor agents thereby altering the pharmacokinetic behavior of the anti-tumor agents and overcoming the toxic side effects. A protein ligand is a protein molecule that exhibits specific binding of high affinity for another molecule, for example, epidermal growth factor (EGF)is a ligand which specifically binds epidermal growth factor receptor(EGFR)on cellular surfaces with high affinity. Protein ligands are often internalized into the cell upon binding to their receptors, thus ligands can be used as vectors to carry cytotoxic molecules specifically into target cells, such as tumor cells. Prior artisans have experimented with a variety of compositions containing ligands linked to anti-tumor agents in an effort to devise an efficient technique for targeting anti-tumor agents to malignant cells, while sparing non-diseased cells. As will be discussed in greater detail in the following section, these experiments have included targeting of individual receptors including vascular endothelial growth factor receptor (VEGFR), epidermal growth factor receptor (EGFR) and the transferrin receptor.

Researchers have attempted to destroy a tumor mass by targeting the tumor vasculature through the vascular endothelial growth factor receptor (VEGFR), for example by attachment of cytotoxic molecules to VEGF (Veenendaal et al. PNAS USA 99(12):7866-7871 2002 and Wild et al. British Journal of Cancer 83(8):1077-1083 2000). In order for a tumor to grow, new blood vessels are required to provide nutrients and to remove waste. Tumor cells secret growth factors to induce the formation of new blood vessels. These newly formed blood vessels are characterized by the expression of surface molecules that are not present on resting endothelium, for example vascular endothelial growth factor receptor (VEGFR). The VEGFR is internalized into the cell upon binding to its ligand, vascular endothelial growth factor (VEGF). Thus, VEGF is functional as a vector to carry cytotoxic molecules into the non-resting endothelial cells in order to induce a tumor-localized vascular collapse leading to necrosis of tumor cells and subsequently a reduction in tumor mass.

Researchers have attempted to destroy a tumor mass by targeting the tumor cells directly through receptors expressed specifically or overexpressed on tumor cells such as epidermal growth factor receptor(EGFR), for example by attachment of cytotoxic molecules to EGF (Uckun et al. Clinical Cancer Research 4:901-912 1998). The EGFR has been identified as a cell surface receptor that is overexpressed on many types of tumor cells. The EGFR is internalized into the cell upon binding to its ligand, epidermal growth factor (EGF). Thus, EGF is functional as a vector to carry cytotoxic molecules into the tumor cells.

Researchers have attempted to destroy a tumor mass by targeting cell surface receptors associated with proliferating cells such as the transferrin receptor, for example, by attachment of cytotoxic molecules to transferrin (U.S. Pat. No. 4,886,780 and U.S. Pat. No.5,792,458). Transferrin is a vertebrate glycoprotein that functions to bind and transport iron. Uptake of iron is mediated in each individual cell by expression of the transferrin receptor. After binding to iron saturated transferrin, the transferrin receptor is internalized to provide the cell with a source of iron. Cells that are actively growing and proliferating show an increased iron requirement, thus these cells also show an increased expression of transferrin receptors. Accordingly, the number of transferrin receptors expressed on the cell surface correlates with cellular proliferation; the highest number expressed on actively growing cells and the lowest number expressed on resting cells. Within the tumor tissue, both the tumor cells and the endothelial cells of the tumor vasculature are actively growing and both show an increased expression of transferrin receptors.

Although targeting each of these receptors (VEGFR, EGFR and transferrin receptor) individually with a composition containing a ligand linked to an anti-tumor agent has been shown to have some degree of efficacy in the treatment of cancerous disease, the efficacy of these compositions is not sufficient to significantly increase the selectivity and therapeutic index of anti-tumor agents.

The present inventor has devised unique integrated moieties containing multiple ligands in linkage with at least one anti-tumor agent. One such moiety contains vascular endothelial growth factor (VEGF) and at least one anti-tumor agent operatively linked to transferrin wherein said VEGF binds VEGF receptors on endothelial cell surfaces of intratumoral blood vessels and said transferrin binds transferrin receptors on cell surfaces of tumor cells and on cell surfaces of intratumoral blood vessels. Another such moiety contains epidermal growth factor (EGF) and at least one anti-tumor agent operatively linked to transferrin wherein said EGF binds EGF receptors on cell surfaces of tumor cells and said transferrin binds transferrin receptors on cell surfaces of tumor cells and on cell surfaces of intratumoral blood vessels. Use of these compositions for cancer therapeutics enables selective concentration of anti-tumor agents to the tumor cells and associated tumor vasculature and simultaneous reduction of the concentration of the anti-tumor agents in non-diseased tissues. Thus, use of these compositions for cancer therapeutics also enables both an increase in the therapeutic index of anti-tumor agents and a reduction in toxicity of anti-tumor agents which represents a difference in kind as compared to the therapeutic index and toxicity of compositions available in the prior art.

Although researchers have heretofore utilized therapeutic methods involving compositions containing a ligand linked to an anti-tumor agent that targets an individual receptor, they have failed to produce a therapeutic method involving a composition capable of selectively concentrating the anti-tumor agents to the tumor cells and associated tumor vasculature while simultaneously reducing the concentration of the anti-tumor agent in non-diseased tissues. What is lacking in the art is a therapeutic method involving a composition capable of selectively concentrating anti-tumor agents to the tumor cells and associated tumor vasculature while simultaneously reducing the concentration of anti-tumor agents in non-diseased tissues.

### DESCRIPTION OF THE PRIOR ART

As is referred to above, prior artisans have experimented with a variety of compositions containing ligands linked to anti-tumor agents in an effort to devise an efficient technique for targeting anti-tumor agents to malignant cells while sparing non-diseased cells. Representative examples include:

U.S. Pat. No. 5,122,368 (Greenfield et al.) discloses conjugates comprising a ligand, such as EGF or transferrin, linked to an anthracycline antibiotic useful for the elimination of a targeted cell population. In the conjugate of Greenfield et al. the ligand is linked to the anthracycline antibiotic through an acid-sensitive hydrazone bond that allows for the release of the anthracycline antibiotic in the acidic environment of the target cell. The conjugates disclosed by Greenfield et al. involve a single ligand targeting an anti-tumor agent to a single receptor.

U.S. Pat. No. 6,214,345 (Firestone et al.) discloses conjugates comprising a ligand, such as transferrin or EGF, linked to a cytotoxic drug useful for the treatment of tumors and other diseases. In the conjugate of Firestone et al. the ligand is linked to the cytotoxic drug through a peptide linker and connector enabling the conjugates to be selectively activatible at the site of the tumor by lysosomal enzymes. The conjugates disclosed by Firestone et al. involve a single ligand targeting an anti-tumor agent to a single receptor.

Lutsenko et al. (Journal of Drug Targeting 10(7):567-571 2002 and Biochemistry 65(11):1299-1304 2000) disclose conjugates comprising an EGF ligand, linked to doxorubicin or carminomycin useful for the treatment of tumors. The conjugates disclosed by Lutsenko et al. involve a single ligand targeting an anti-tumor agent to a single receptor.

Arencibia et al. (International Journal of Oncology 19(3):571-577 2001) discloses conjugates comprising a ligand which is an analog of luteinizing hormone-releasing hormone (LHRH) linked to doxorubicin useful in chemotherapy of ovarian cancers expressing LHRH receptors. The conjugates disclosed by Arencibia et al. involve a single ligand targeting an anti-tumor agent to a single receptor.

Wang et al. (Anticancer Research 20(2A):799-808 2000) discloses conjugates comprising a ligand (transferrin saturated with ferric chloride or gallium nitrate) linked to doxorubicin useful for overcoming multi-drug resistance in breast cancer cells. The conjugates disclosed by Wang et al. involve a single ligand targeting an anti-tumor agent to a single receptor.

Munns et al. (British Journal of Urology 82(2):284-289 1998) discloses conjugates comprising a ligand (transferrin) linked to adriamycin expected to be useful for overcoming multi-drug resistance in bladder cancer cell lines. The conjugates disclosed by Munns et al. involve a single ligand targeting an anti-tumor agent to a single receptor. However, the conjugate disclosed by Munns et al. was unsuccessful at achieving the goal of overcoming multi-drug resistance in bladder cancer cell lines (see abstract of Munns et al.).

Kratz et al. (Journal of Pharmaceutical Science 87(3):338-346 1998) discloses conjugates comprising a ligand (transferrin) linked to doxorubicin useful for treatment of cancers. Kratz et al. synthesized their conjugates by conjugation of thiolated human serum transferrin with four maleimide derivitives of doxorubicin that differed in the stability of the chemical linkage between the drug and the spacer. The conjugates disclosed by Kratz et al. involve a single ligand targeting an anti-tumor agent to a single receptor.

An important distinction between the instant invention and the prior art involves the source of experimental tumors. Tumors grown in immunodeficient mice which are derived from cell lines often develop vasculature of murine origin. The VEGF isoform used in the instant invention is of human origin and thus will react only with VEGF receptors on endothelial cells of human origin. The compositions used in the methods of the instant invention containing VEGF would be ineffective if used against murine blood vessels. The tumors targeted in the experiments described herein are all derived from human surgical specimens and exhibit vasculature of human origin (see FIG. 3). In contrast, the tumors which are targeted in the experiments disclosed in the above-referenced prior art are all derived from cell lines and hence would exhibit blood vessels of murine origin. Thus, the instant invention provides an improved model system for targeting angiogenesis in human tumors.

Additionally, it is important to note that all of the compositions used in the methods disclosed in the above references involve a single ligand targeting an anti-tumor agent to a single receptor, this is in contrast to the compositions used in the methods of the instant invention wherein multiple ligands target an anti-tumor agent to multiple receptors located on both tumor cells and on endothelial cells of the associated tumor vasculature. None of the above references disclose or suggest a method using a composition capable of selectively concentrating anti-tumor agents to the tumor cells and associated tumor vasculature while simultaneously reducing the concentration of anti-tumor agents in non-diseased tissues.

### SUMMARY OF THE INVENTION

The instant invention provides methods involving compositions capable of selectively concentrating anti-tumor agents to the tumor cells and associated tumor vasculature while simultaneously reducing the concentration of the anti-tumor agent in non-diseased tissues. The compositions used in the methods of the instant invention contain multiple ligands capable of targeting an anti-tumor agent to multiple receptors located on both tumor cells and on endothelial cells of the associated tumor vasculature. The multi-targeting ability of the compositions used in the methods of the instant invention enables delivery of anti-tumor agents in a manner which simultaneously increases their therapeutic index and reduces their toxicity.

The composition used in the methods of the instant invention contains a ligand such as human vascular endothelial growth factor (VEGF)or human epidermal growth factor (EGF)and at least one anti-tumor agent operatively linked to a human transferrin ligand, wherein said human VEGF binds to VEGF receptors on endothelial cell surfaces of intratumoral blood vessels, said human EGF binds to human EGF receptors when present on cell surfaces of tumor cells and said human transferrin binds human transferrin receptors on endothelial cell surfaces of intratumoral blood vessels and cell surfaces of tumor cells. FIG. 1 shows a schematic diagram of the compositions used in the methods described herein.

As used herein, the term “ligand” refers to a molecule that exhibits specific binding of high affinity for another molecule and upon binding with that molecule is internalized into the cellular interior. An illustrative, albeit non-limiting example of how the term “ligand” is used in the context of the instant specification is a protein ligand binding to a cell surface receptor, such as EGF binding to the EGFR.

As used herein, the term vascular endothelial growth factor (VEGF) encompasses VEGF and isolated peptide fragments or biologically active portions thereof, analogues of VEGF and any biologically active portion thereof and any molecules and portions of molecules having the biological activity of VEGF.

As used herein, the term epidermal growth factor (EGF) encompasses EGF and isolated peptide fragments or biologically active portions thereof, analogues of EGF and any biologically active portion thereof and any molecules and portions of molecules having the biological activity of EGF.

As used herein, the term transferrin encompasses transferrin and isolated peptide fragments or biologically active portions thereof, analogues of transferrin and any biologically active portion thereof and any molecules and portions of molecules having the biological activity of transferrin.

As used herein, the term “bioactivity” refers to the ability of a ligand to bind to its complementary receptor thus enabling internalization of the ligand into the cellular interior.

As used herein, the term “biologically active portion” refers to the portion of a ligand that has the ability to bind to its complementary receptor thus enabling internalization of the ligand into the cellular interior.

As used herein, the term “anti-tumor agent” refers to any substance that is capable of inhibiting the proliferation of or killing cells of tumor tissues.

With regard to the compositions used in the methods of the instant invention, vascular endothelial growth factor (VEGF) acts as a vector for delivery of anti-tumor agents to the endothelial cells of the tumor vasculature and epidermal growth factor (EGF) acts as a vector for delivery of anti-tumor agents to the tumor cells. Transferrin acts as a dual-functioning vector for delivery of anti-tumor agents to both the tumor cells and the endothelial cells of the tumor vasculature.

Multiple doses are administered over a period of time for the purpose of treatment and extend the periods of treatment normally seen with anti-tumor agents since the methods of the instant invention reduce side effects. The period of time between doses is selected based upon the needs of the host receiving the treatment. Illustrative, albeit non-limiting examples of periods of time allowed between doses are hours, days and weeks. A particularly preferred period of time between doses is one week, the use of which is illustrated in the examples herein. A therapeutic dose is administered each selected period of time until a statistically significant inhibition of tumor growth is achieved. The amount of inhibition is determined by comparison of tumor growth in treated animals with tumor growth in control animals which have not received treatments.

Since it functions as a transporter of iron, the transferrin molecule has iron-binding sites. Radioactive ions can be bound in the iron-binding sites of the transferrin ligand of the compositions used in the methods of the instant invention for an additional cytotoxic effect. Illustrative, albeit non-limiting examples of radionuclides known and commonly used in the art for radioactive labeling are ¹²³I, ¹²⁵I, ¹³⁰I, ¹³¹I, ¹³³I, ¹³⁵I, ⁴⁷Sc, ⁷²As, ⁷²Se, ⁹⁰Y, ⁸⁸, ⁹⁷Ru, ¹⁰⁰Pd, ^{101m}Rh, ¹¹⁹Sb, ¹²⁸Ba, ¹⁹⁷Hg, ²¹¹At, ²¹²Bi, ¹⁵³Sm, ¹⁶⁹Eu, ²¹²Pb, ¹⁰⁹Pd, ¹¹¹In, ⁶⁷Ga, ⁶⁸Ga, ⁶⁷Cu, ⁷⁵Br, ⁷⁶Br, ⁷⁷Br, ^{99m}Tc, ¹¹C, ¹³N, ¹⁵O and ¹⁸F. A particularly preferred radiolabel is ¹¹¹ In.

The compositions used in the methods of the instant invention can be added to a pharmacologically effective amount of a carrier to provide pharmaceutical compositions for administration to an animal host, including administration to a human patient. Illustrative, albeit non-limiting examples of carriers known in the art and suitable for use with the instant invention are water, saline solutions and dextrose solutions. A particularly preferred carrier is saline, the use of which is illustrated in the examples herein.

Any anti-tumor agent is considered to be encompassed within the scope of the instant invention. Illustrative, albeit non-limiting examples are discussed above the “Background of the Invention” section of the instant specification. A particularly preferred class of anti-tumor agents for use within the context of the methods of the instant invention is the anti-tumor antibiotics, the use of which is illustrated in the examples herein. The anti-tumor antibiotic doxorubicin was used in the experimental examples herein described. However, it is noted that the use of the methods of the instant invention to deliver doxorubicin is an illustrative example only and is not intended to limit the methods to delivery of doxorubicin. The methods of the instant invention can be used to deliver any anti-tumor agent to any host having a tumor.

Accordingly, it is an objective of the instant invention to provide a method for inhibiting the growth of tumor tissue, said method comprising the steps of:(a) administering a biologically effective amount of a compound to a host having a tumor, said compound comprising human vascular endothelial growth factor (VEGF) and at least one anti-tumor agent operatively linked to human transferrin, and (b) repeating said administering of step (a) over a period of time until a statistically significant inhibition of tumor growth is achieved.

It is another objective of the instant invention to provide a method for inhibiting the growth of tumor tissue, said method comprising the steps of: (a) administering a biologically effective amount of a compound to a host having a tumor, said compound comprising human vascular endothelial growth factor (VEGF) and at least one anti-tumor agent operatively linked to radiolabeled human transferrin, and (b) repeating said administering of step (a) over a period of time until a statistically significant inhibition of tumor growth is achieved.

It is another objective of the instant invention to provide a method for inhibiting the growth of tumor tissue, said method comprising the steps of: (a) administering a biologically effective amount of a conjugate to a host having a tumor, said conjugate consisting essentially of human vascular endothelial growth factor (VEGF)and at least one anti-tumor agent operatively linked to human transferrin, and (b) repeating said administering of step (a) over a period of time until a statistically significant inhibition of tumor growth is achieved.

It is yet another objective of the instant invention to provide a method for inhibiting the growth of tumor tissue, said method comprising the steps of: (a) administering a biologically effective amount of a conjugate to a host having a tumor, said conjugate consisting essentially of human vascular endothelial growth factor (VEGF) and at least one anti-tumor agent operatively linked to radiolabeled human transferrin, and (b) repeating said administering of step (a) over a period of time until a statistically significant inhibition of tumor growth is achieved.

It is another objective of the instant invention to provide a method for inhibiting the growth of tumor tissue, said method comprising the steps of: (a) administering a biologically effective amount of a compound to a host having a tumor, said compound comprising human epidermal growth factor (EGF) and at least one anti-tumor agent operatively linked to human transferrin, and(b) repeating said administering of step (a) over a period of time until a statistically significant inhibition of tumor growth is achieved.

It is another objective of the instant invention to provide a method for inhibiting the growth of tumor tissue, said method comprising the steps of: (a) administering a biologically effective amount of a compound to a host having a tumor, said compound comprising human epidermal growth factor (EGF) and at least one anti-tumor agent operatively linked to radiolabeled human transferrin, and (b) repeating said administering of step (a) over a period of time until a statistically significant inhibition of tumor growth is achieved.

It is yet another objective of the instant invention to provide a method for inhibiting the growth of tumor tissue, said method comprising the steps of: (a) administering a biologically effective amount of a conjugate to a host having a tumor, said conjugate consisting essentially of human epidermal growth factor (EGF)and at least one anti-tumor agent operatively linked to human transferrin, and (b) repeating said administering of step (a) over a period of time until a statistically significant inhibition of tumor growth is achieved.

It is another objective of the instant invention to provide a method for inhibiting the growth of tumor tissue, said method comprising the steps of: (a) administering a biologically effective amount of a conjugate to a host having a tumor, said conjugate consisting essentially of human epidermal growth factor (EGF) and at least one anti-tumor agent operatively linked to radiolabeled human transferrin, and (b) repeating said administering of step (a) over a period of time until a statistically significant inhibition of tumor growth is achieved.

Other objectives and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE FIGURES

FIG. 1 shows a diagrammatic presentation of the compositions used in the methods described herein.

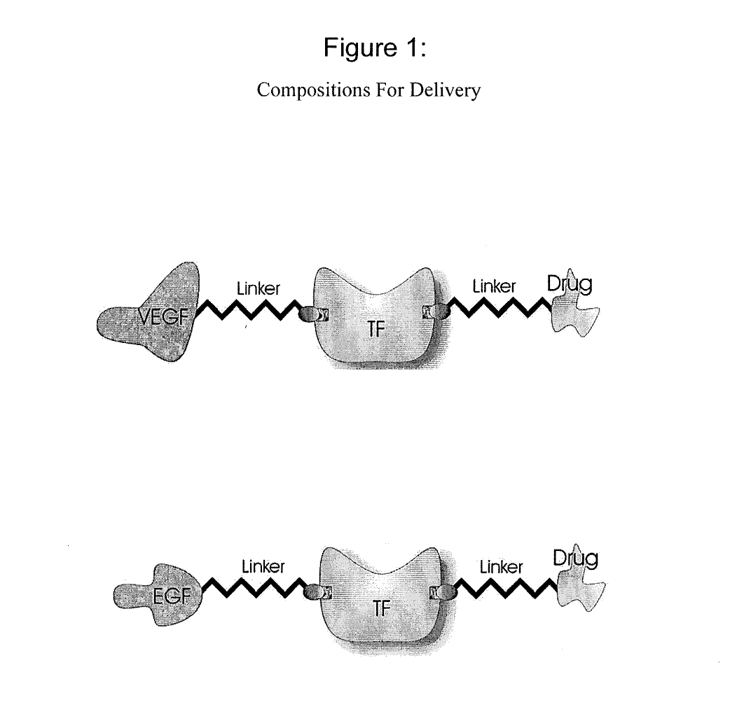

FIGS. 2A-2B show immunohistochemistry of BCBM specific for EGFR (epidermal growth factor receptor). FIG. 2A shows a histologic section stained with antibody (TS40) specific for the human cell surface EGFR. FIG. 2B is a micrograph showing an isolated EGFR+ breast cancer cell in the bone marrow.

FIG. 3 is a micrograph showing blood vessels of human origin in the BCBM tumors in SCID mice.

FIG. 4 shows a graphical presentation comparing the inhibition of breast cancer growth achieved by carrying out methods of the instant invention.

FIG. 5 shows a graphical presentation of Breast Cancer Bone Metastatsis (BCBM) volumes in SCID mice.

### DEFINITIONS

The following list defines terms, phrases and abbreviations used throughout the instant specification. Although the terms, phrases and abbreviations are listed in the singular tense the definitions are intended to encompass all grammatical forms.

As used herein, the abbreviation “EGF” refers to epidermal growth factor.

As used herein, the abbreviation “EGFR” refers to epidermal growth factor receptor.

As used herein, the abbreviation “VEGF” refers to vascular endothelial growth factor.

As used herein, the abbreviation “VEGFR” refers to vascular endothelial growth factor receptor.

As used herein, the abbreviation “BCBM” refers to breast cancer bone metastatsis.

As used herein, the abbreviation “PEG” refers to polyethylene glygol.

As used herein, the abbreviation “TF” refers to transferrin.

As used herein, the abbreviation “SA” refers to streptavidin.

As used herein, the abbreviation “TF/SA” refers to a composition comprising transferrin linked to streptavidin.

As used herein, the abbreviation “MBS” refers to m-maleimidobenzoyl N-hydroxysuccinimide ester.

As used herein, the abbreviation “HPLC” refers to high performance liquid chromatography.

As used herein, the abbreviation “RP-HPLC” refers to reverse phase high performance liquid chromatography.

As used herein, the abbreviation “NHS” refers to N-hydroxysuccinimide.

As used herein, the abbreviation “TFA” refers to trifluoroacetic acid.

As used herein, the abbreviation “PBS” refers to phosphate buffered saline.

As used herein, the abbreviation “SCID” refers to a type of transgenic mouse that is severe combined immuno-deficient.

As used herein, the abbreviation “5-FU” refers to the anti-tumor agent 5-fluorouracil.

As used herein, the abbreviation “FUDR” refers to the anti-tumor agent floxuridine.

As used herein, the abbreviation “6-MP” refers to the anti-tumor agent 6-mercaptopurine.

As used herein, the term “anti-tumor agent” refers to any substance that is capable of inhibiting the proliferation of or killing cells of tumor tissues.

As used herein, the term “selective delivery” is defined as delivery which is targeted to a specific cell type for the purpose of avoiding uniform or even delivery to all cell types.

As used herein, the term “selective concentration” is defined as concentrating a substance, such as an anti-tumor agent, to a specific area for the purpose of avoiding uniform or even concentration of a substance in all areas.

As is used herein, the term “dose” is defined as the amount of a substance administered at one time. A dose should be administered in mg per kg of body weight of the host to be which it is to be administered.

As is used herein, the term “therapeutic index” is defined with regard to dose and indicates safety of a substance. A dose is administered in an amount having a specified effect on a stated fraction of experimental animals tested. The therapeutic index is defined by the fraction LD₅₀/ED₅₀ wherein LD₅₀ represents the dose causing death in 50% of experimental animals and ED₅₀ represents the dose at which 50% of the experimental animals show an effect. The methods of the instant invention concentrate anti-tumor agents to the tumor tissue and simultaneously reduce concentration of anti-tumor agents in non-diseased tissues, resulting in increased death of tumor tissue and decreased death of non-diseased tissues. Thus the therapeutic index of anti-tumor agents is increased by the methods of the instant invention. (see Concise Encyclopedia Biochemistry and Molecular Biology, Third Edition, revised and expanded by Thomas A. Scott and E. Ian Mercer, Walter de Gruyter publisher, Berlin and New York, 1997, page 185, for a discussion of dose and therapeutic index).

As used herein, the term “ligand” refers to a molecule that exhibits specific binding of high affinity for another molecule and upon binding with that molecule is internalized into the cellular interior. An illustrative, albeit non-limiting example of how the term “ligand” is used in the context of the instant specification is a protein ligand binding to a cell surface receptor, such as EGF binding to the EGFR.

As used herein, the term “receptor” refers to a molecule that exhibits specific binding of high affinity for its complementary ligand. An illustrative, albeit non-limiting example of how the term “receptor” is used in the context of the instant specification is a cell surface receptor binding to a ligand, such as the EGFR binding the EGF.

As used herein, the term “complementary receptor” refers to the receptor a ligand specifically binds with high affinity, for example, the EGFR is the complementary receptor for EGF.

As used herein, the term “target” refers to a specific molecule expressed on the cellular surface such as a receptor to which a specific moiety can be directed, for example the EGFR is a target for EGF.

As used herein, the term “targeting agent” refers to a specific molecule that binds to a complementary molecule expressed on the cellular surface such as a ligand, for example EGF is a targeting agent for the EGFR.

As used herein, the phrase “multi-targeted” refers to the ability of a therapeutic protocol to target at least two disease elements, for example, the methods of the instant invention can be used to target an entire tumor mass by using EGF to target the tumor cells (or by using VEGF to target the endothelial cells of the tumor vasculature) and by using transferrin to target both the tumor cells and the endothelial cells of the tumor vasculature.

As used herein, the phrase “disease elements” refers to the separate targets or elements that contribute to result in an entire disease state, for example, malignant cells and endothelial cells are each separate disease elements in cancer pathology.

As used herein, the term “VEGF” refers to a glycosylated polypeptide that serves as a mitogen to stimulate vascular development. VEGF imparts activity by binding to vascular endothelial cell plasma membrane-spanning tyrosine kinase receptors (VEGFR's) which then activates signal transduction.

As used herein, the term “VEGFR” refers to a vascular endothelial cell plasma membrane-spanning tyrosine kinase receptor which binds VEGF thus exerting a mitogenic signal to stimulate vascularization of tissues.

As used herein, the term vascular endothelial growth factor (VEGF) encompasses VEGF and isolated peptide fragments or biologically active portions thereof, analogues of VEGF and any biologically active portion thereof and any molecules and portions of molecules having the biological activity of VEGF.

As used herein, the term “EGF” refers to a mitogenic polypeptide that exhibits growth stimulatory effects for epidermal and epithelial cells. EGF imparts activity by binding to epidermal and/or epithelial cell plasma membrane-spanning tyrosine kinase receptors (EGFR's) which then activates signal transduction.

As used herein, the term “EGFR” refers to a epidermal and/or epithelial cell plasma membrane-spanning tyrosine kinase receptor which binds EGF thus exerting a mitogenic signal.

As used herein, the term epidermal growth factor (EGF) encompasses EGF and isolated peptide fragments or biologically active portions thereof, analogues of EGF and any biologically active portion thereof and any molecules and portions of molecules having the biological activity of EGF.

As used herein, the term “transferrin” refers to a vertebrate glycoprotein that functions to bind and transport iron.

As used herein, the term “transferrin receptor” refers to a receptor expressed on the surface of cells functioning to capture and bind iron saturated transferrin. Expression of the transferrin receptor is increased in cells which are actively proliferating.

As used herein, the term transferrin encompasses transferrin and isolated peptide fragments or biologically active portions thereof, analogues of transferrin and any biologically active portion thereof and any molecules and portions of molecules having the biological activity of transferrin.

As used herein, the term “host” refers to any animal suspected of having or having a tumor, including a human patient.

As used herein, the term “tumor tissue” refers to all of the cellular types which contribute to formation of a tumor mass, including tumor cells and endothelial cells, for example, the tumor tissue includes tumor cells and blood vessels.

As used herein, the term “tumor mass” refers to a foci of tumor tissue.

As used herein, the term “inhibition” refers to retarding the growth of a tumor mass.

As used herein, the term “bioactivity” refers to the ability of a ligand to bind to its complementary receptor thus enabling internalization of the ligand into the cellular interior.

As used herein, the term “biologically active portion” refers to the portion of a ligand that has the ability to bind to its complementary receptor thus enabling internalization of the ligand into the cellular interior.

As used herein, the phrase “biologically effective amount” refers to the composition used in the method of the instant invention administered to a host having a tumor in an amount sufficient for the composition to carry outs its bioactivity and thus inhibit the growth of tumor tissue.

As used herein, the phrases, “tumor vasculature”, “tumor endothelium” and “tumor vessels” all refer to the vessels which supply the tumor tissue with blood.

As used herein, the term “angiogenesis” refers to the process by which tissues become vascularized. Angiogenesis involves the proteolytic degradation of the basement membrane on which the endothelial cells reside followed by the chemotactic migration and mitosis of the endothelial cells to support a new capillary shoot.

As used herein, the term “linker” refers to the molecules which join the ligands of the composition used in the methods of the instant invention together to form a single composition; for example, EGF-PEG attached to biotin links streptavidin attached to transferrin.

As used herein, the phrase “operatively linked” means that the linkage does not destroy the functions of each of the separate elements of the composition used in the methods of the instant invention, for examples when linked together by a linker to form the single composition used in the methods of the instant invention the ligands retain the ability to bind their complementary receptors.

As used herein, the term “carrier” refers to a pharmaceutically inert substance that facilitates delivery of an active agent to a host, for example, as is shown in the experiments described herein, saline functions as a carrier for delivery of the compositions used in the methods of the instant invention to the mouse host.

As used herein, the phrase “pharmacologically effective amount of a carrier” refers to an amount of a carrier that is sufficient to effectively deliver an active agent to a host.

As used herein, the term “pharmaceutical composition” refers to the compositions used in the methods of the instant invention combined with a pharmacologically effective amount of a carrier.

The phrases “tumor endothelium”, “tumor vasculature” and “tumor vessels” are used interchangeably herein.

The terms “tumor cell”, “neoplastic cell” and “cancer cell” are used interchangeably herein.

As used herein, the term “compound” refers to a substance containing at least two distinct elements to which an unlimited number of other elements can be added.

As used herein, the term “conjugate” refers to a substance containing at least two distinct elements and a defined number of additional elements.

As used herein, the term “composition” is intended to encompass both a compound and a conjugate.

### DETAILED DESCRIPTION OF THE INVENTION

### Experimental Procedures

Sequences

The following nucleic acid sequences and corresponding amino acid sequences were used to generate the DNA and polypeptides used in the experiments described herein. Homo sapiens (human) VEGF165 (vascular endothelial growth factor isoform 165)nucleic acid sequence is disclosed as SEQ ID NO:1 and translates into VEGF165 protein disclosed as amino acid sequence SEQ ID NO:2. Homo sapiens (human) transferrin nucleic acid sequence is disclosed as SEQ ID NO:3 and translates into transferrin protein disclosed as amino acid sequence SEQ ID NO:4. Homo sapiens (human) EGF (epidermal growth factor) nucleic acid sequence is disclosed as SEQ ID NO:5 and translates into EGF protein disclosed as amino acid sequence SEQ ID NO:6.

Linkers

When assembling compositions from multiple elements, elements are either linked directly through chemical conjugation (for example through reaction with an amine or sulfhydryl group) or are linked indirectly through molecules termed linkers. When selecting a linker it is important to choose the appropriate length and flexibility of linker in order to reduce steric hindrance between the elements of the compositions. For example, if an element of a composition is brought into close physical proximity of another element by linkage, the function of either or both elements can be affected. Each element of the composition must retain its bioactivity, for example in the composition used in the methods of the instant invention, each ligand must retain its ability to bind to its complementary receptor after linkage with the other ligands of the composition. Illustrative, albeit non-limiting examples of linkers are glycols, alcohols and peptides. Particularly preferred linkers are PEG (polyethylene glycol) and the peptide linker shown as SEQ ID NO:8 (use of each of these linkers is illustrated in the examples described herein).

Crosslinking of VEGP (AND EGP) to a Biotinylated-Polylinker

EGF and VEGF are crosslinked to a biotinylated polylinker by carrying out the following protocol. The polylinker used consists of 15 amino acid residues shown as SEQ ID NO:8. The cDNA sequence encoding this polylinker is shown as SEQ ID NO:7. The first glycine residue at the N-terminal was biotinylated. EDC (1-Ethyl-3-(3-Dimethylaminopropyl) carbodiimide Hydrochloride) and NHS (N-Hydroxysuccinimide) were equilibrated to room temperature. 0.4 mg of EDC and 0.6 mg of NHS were added to 1 mg/ml of the polylinker peptide solution (in activation buffer: 0.1 M MES (2-[N-morpholino] ethane sulfonic acid), 0.5 M NaCl, pH 6.0) to a final concentration of EDC and NHS of 2 mM and 5 mM respectively. The reaction mixture was then held for 15 minutes at room temperature. 1.4 μl of 2-mercaptoethanol was then added (to a final concentration of 20 mM). The reaction mixture was then run through P2 gel filtration mini-column and eluted by the activation buffer. Fractions containing the protein were then pooled together. Equal mole:mole ratios of either VEGF or EGF protein were added to the pooled fractions and reacted for 2 hours at room temperature. Hydroxylamine was added to a final concentration of 10 mM and the VEGF-linker or EGF-linker was purified by P2 gel filtration mini-column.

Synthesis of TF/SA Composition

8.84mg of transferrin (TF) was thiolated by adding a 5-fold molar excess of 2-Iminothiolane hydrochloride (Traut's reagent) in pH 8.0, 0.16 M borate. Following 90 minutes at room temperature, the thiolated TF was desalted and concentrated by Centricon microconcentrators. Ellman's reagent (Pierce) was then used to demonstrate that a single thiol group was inserted on the surface of TF. 7 mg of streptavidin (SA) (in PBS) was activated by adding to a 20:1 molar ratio of m-maleimidobenzoyl N-hydroxysuccinimide ester (MBS)(stock at 1 mg/ml in dimethylformamide). After 20 minutes, the activated SA was desalted on a microconcentrator and immediately, the activated SA was added to a 10 molar excess of thiolated TF. They were mixed and then incubated at room temperature for 3 hours. Purification of the TF/SA composition was done by HPLC using TSK-G3000 column. The number of biotin binding sites per TF/SA composition was determined with ³H-biotin binding assay.

Conjugation of VEGF-Linker-Biotin (and EGF-Linker-Bioton) to TF-SA and ¹¹¹In-Labeling

VEGF-Linker-Biotin and EGF-Linker-Biotin are added to TF/SA by carrying out the following protocol. The composition of VEGF-Linker-biotin (or EGF-Linker-biotin) and TF/SA was prepared by mixing 5 nmol of VEGF-Linker-biotin (or 5nmol of EGF-Linker-biotin) with 8 nmol of TF/SA (1:1.6 molar ratio). HPLC was then used to purify the VEGF-Linker-biotin-TF-SA composition (or EGF-Linker-biotin-TF-SA composition). The reaction mixture was then applied to a TSK-gel G3000 SW_{XL} HPLC gel filtration column, followed by elution in 0.01 M Na₂HPO_{4/}0.15 M NaCl/pH 7.4/0.05% Tween-20 at a flow rate of 0.5 mL/min for 40 minutes, and 0.5 mL fractions were collected. 2 mCi ¹¹¹In acetate was mixed with the composition in 10 mM HEPES, 15 mM NaHCO3 pH 7.4 buffer for 1 hour at room temperature.

Free ¹¹¹In was separated from bound ones by running the reaction volume through P2 (BioRad) size-exclusion chromatography using a mini-column and the 111In bound-protein was eluted with pH 7.4 10 mM HEPES, 15 mM NaHCO3 buffer. Fractions collected (100 μl) were measured for radioactivity and fractions containing the protein were combined and the specific radioactivity of proteins was determined. labeled proteins were used immediately.

Conjugation of VEGF (and EGF) to Peg3400-Biotin

Alternatively to linkage with a peptide linker, VEGF and EGF can also be linked to transferrin using PEG by carrying out the following protocol. NHS-PEG3400-biotin was obtained from Shearwater Polymers (Huntsville, Ala.), where NHS=N-hydroxysuccinimide and PEG3400=poly(ethylene glycol) of 3400 Da molecular mass. NHS-PEG3400-biotin (20 nmol in 310 μl of 0.05 M NaHCO3) was added in a 1:1 molar ratio to either VEGF or EGF (16 nmol in 250 μl of 0.05 M NaHCO3) followed by incubation at room temperature for 60 minutes. The mixture was then applied to two Sepharose 12 HR 10/30 FPLC columns in series, followed by the elution in 0.01 M NaH2PO4/0.15 M NaCl/pH 7.5 at a flow rate of 0.7 mL/minute for 120 minutes. Fraction(s) that contained VEGF or EGF bound to PEG3400-biotin moiety were pooled together.

Conjugation of VEGF-PEG3400-Biotin (and EGF-PEG3400-Biotin) to TF-SA and ¹¹¹In-Labeling

Following reaction of EGF and/or VEGF with NHS-PEG3400-biotin and transferrin with streptavidin, both compositions were purified by HPLC. The EGF (and/or VEGF)-NHS-PEG3400-biotin and TF/SA compositions were then mixed (1:1.6 molar ratio). The compositions EGF (and/or VEGF)-NHS-PEG3400-biotin-TF-SA were purified by HPLC and labeled with ¹¹¹In by mixing with ¹¹¹In acetate and purified on a P-2 size-exclusion mini-column. The specific activity of ¹¹¹In-EGF (and/or VEGF)-PEG3400-biotin-TF-SA compositions were about 100-400 mCi/mg.

Conjugation of Doxorubicin to VEGF-Transferrin and EGF-Transferrin

It is noted that the following protocol for addition of the anti-tumor agent is useful for compositions containing PEG linkers, peptide linkers, radiolabeled transferrin or unlabeled transferrin. Conjugation of Doxorubicin to EGF-transferrin (and VEGF-transferrin) was performed as follows; a solution containing 20 mg human EGF-transferrin (or VEGF-transferrin) and 6 mg of Doxorubicin hydrochloride (Farmitalia Co., Milan, Italy) in 2 ml of 0.1 M phosphate buffered saline (PBS), pH 7.0 was added dropwise to 1.0 ml of an aqueous solution of 0.25% glutaraldehyde (BDH Chemicals Ltd., Poole, England) at room temperature. After 2 hours incubation at room temperature in the dark, 1.0 ml of 1 M ethanolamine (Sigma USA) pH 7.4., was added and the mixture was incubated at 4° C. overnight. The mixture was then centrifuged at 1,000g for 15 minutes and the supernatant was collected and chromatographed through a column of Sepharose CL-6B (Pharmacia Fine Chemicals, Uppsala, Sweden) and equilibrated in 0.15 M PBS, pH 7.2. EGF-NHS-PEG3400-Biotin-TF-SA-Doxorubicin (or VEGF-NHS-PEG3400-Biotin-TF-SA-Doxorubicin) fractions were collected. Doxorubicin can also be linked with EGF-transferrin and VEGF-transferrin as described by Kratz et al. (Journal of Pharmaceutical Sciences 87(3):338-346 1998) or Lejeune et al. (Anticancer Research 14(3A):915-919 1994).

Experimental Mice

Severe combined immuno-deficient C.B.-17 scid/scid (SCID) mice were bred and maintained according to the protocol of Sandhu et al. (Critical Reviews in Biotechnology 16(1):95-118 1996). Mice were used when 6-8 weeks old and were pre-treated with a dose of 3 Gy γ-radiation administered from a ¹³⁷CS source (Gamacell, Atomic Energy of Canada Ltd. Commercial Products). The irradiated SCID mice receive intraperitoneal injection of 20 μl ASGM1 sera diluted to 100 μl with saline, 4 hours pre- bone transplantation and every 7 days thereafter for the duration of the experiments.

Experimental Tumors

The methods of the instant invention are effective when used to target either an EGFR+tumor or an EGFR- tumor since the transferrin moiety targets those tumor cells that are EGFR−. A bone metastatic focus of a primary EGFR+ breast tumor was used in the experimental examples herein described. However, it is noted that the use of the methods of the instant invention in breast tumors is an illustrative example only and is not intended to limit the use of the methods to breast tumors. The methods of the instant invention can be carried out in a host having any tumor comprising cells which are positive for the expression of at least one of the cell surface receptors described herein (the transferrin receptor, the EGFR and the VEGFR).

Implantation of Humam Breast Cancer Bone Metastasis in SCID Mice

Breast cancer bone metastasis (BCBM) specimens (n=20, JJ1 to JJ20) were obtained from female patients (age range 40-68 years) undergoing total hip joint replacement due to BCBM mediated bone osteolysis. The majority (70%) of the BCBM used in these experiments were infiltrative ductal carcinoma and each specimen was assigned a number JJ1 to JJ20. Normal cancellous bone was obtained from healthy adult patients (age range 59-80 years) undergoing total hip joint replacement for the treatment of degenerative osteoarthritis. The BCBM was obtained from the proximal femur, morcellized using a rongeur and maintained under sterile conditions in RPMI (1640) medium (Gibco BRL, Burlington Ontario, Canada). Transplantation of the normal bone and BCBM into mice was performed within 2 hours of procurement, under a general anesthetic (intramuscular administration of Xylazine (4 μl/20g mouse), and Ketamine (4 μl/20g mouse) in 40 μl of 0.9% sodium chloride) under sterile conditions. Morcellized normal bone (Bone-SCID mice), and BCBM (BCBM-SCID mice), approximately 0.121 cm³ per mouse, was transplanted subcutaneously over the left flank in SCID mice (n=30).

Tumor Measurement

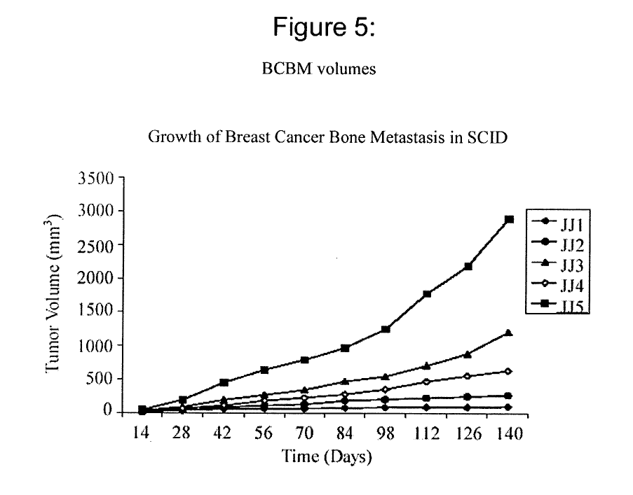

BCBM volumes were measured every 14 days for 20 weeks to assess tumor growth in SCID mice as described by Osborne et al.(Cancer Research 45:584-590 1985). The data shows that in contrast to the similar growth rate of breast cancer cell lines in immunodeficient mice the growth pattern of BCBM specimens varies in SCID mice (see FIG. 5). Results showed JJ5 gave the best growth of the tumor, thus it was chosen as the surgical specimen for use in subsequent in vitro cell studies and in vivo animal experiments.

Cell Culture Studies

Measurement of EGF-¹¹¹In-Labeled Transferrin Composition Binding to Breast CanceR Cells

Breast cancer cells express up to 100-fold higher levels of EGFR than do normal epithelial tissues. EGFR expression in breast cancer bone metastasis biopsies ranged from 1-1300 fmol/mg membrane protein (approximately 400-1,000,000 receptors/cell) and was associated with high relapse rate and poor long term survival. Normal epithelial cells express <10⁴ receptors/cell.

For the normal breast cell line HBL-100, 8000 EGFR/cell has been reported. The expression of EGFR in breast cancer cell lines has a reported range of 800 EGFR/cell for MCF-7 cells to 10⁶ EGFR/cell for MDA-MB-468 cells. The liver is the only normal tissue exhibiting moderate levels of EGFR (8×10⁴ to 3×10⁵ receptors/cell) likely reflecting its role in the elimination of EGF from the blood. Utilizing the Auger electron emitter ¹¹¹In was used in the initial experiments to illustrate the utility of the invention using EGF-¹¹¹In-labeled transferrin compositions. The EGF-¹¹¹In-labeled transferrin (0.25-80 ng) was incubated with 1.5×10⁶ cells/dish JJ5 Breast Cancer (prepared from BCBM JJ5) cells in 1 mL of 0.1% human serum albumin in 35 mm multiwell culture dishes at 37° C. for 30 minutes. The cells were transferred to a centrifuge tube and centrifuged. The cell pellet was separated from the supernatant and counted in a g-scintillation counter to determine bound (B) and free (F) radioactivity. Non-specific binding was determined by conducting the assay in 100 nM hEGF. The kinetics of binding was determined by incubating 1 ng of EGF-¹¹¹In-labeled transferrin composition with 3×10⁶ JJ5 Breast Cancer cells at 37° C. and determining the proportion of radioactivity bound to the cells at various times up to 24 hours. Internalized fraction was measured by determining the proportion of radioactivity which could not be displaced from the cell surface by 100 nM hEGF. Cell-associated binding (surface-binding and intracellular accumulation) was expressed as a percentage of medium radioactivity bound per mg of cell study protein.

The affinity constant for binding of EGF-¹¹¹In-labeled transferrin composition to JJ5 cells was 8×10⁸ L/mol and the number of binding sites was 2.7×10⁶. EGF-¹¹¹In-labeled transferrin composition was rapidly bound by the breast cancer cells and retained for at least 24 hours. Over a 24 hour period at 37° C., <8% was lost from the cells in vitro.

The Growth Inhibition Assay of EGF-¹¹¹In-Labeled Transferring Composition Against JJ5 Breast Cancer Cells

JJ5 breast cancer cells (prepared from BCBM JJ5) expressing approximately 10⁶ epidermal growth factor receptors/cell were incubated with EGF-¹¹¹In-labeled transferrin composition, unlabeled hEGF or ¹¹¹In-oxine, centrifuged to remove free ligand, then assayed and seeded (10¹⁶ cells/dish) into 35 mm culture dishes. Growth medium was added and the cells were cultured at 37° C./5% CO² for 4 days. The cells were then recovered by trypsinization and counted in a hemocytometer. Control dishes contained cells cultured in growth medium containing ¹¹¹In-DTPA or growth medium alone.

The growth inhibition assay of EGF-¹¹¹In-labeled transferrin composition (3.4 pCi/cell) achieved a 83% growth inhibition of the JJ5 cells compared to the medium control, whereas ¹¹¹In oxine (3.5 pCi/cell) which enters all the cells resulted in 91% growth inhibition.

Cytotoxocyty Assay of EGF-¹¹¹In-Labeled Transferrin Composition Against JJ5 Breast Cancer Cells

JJ5 breast cancer cells were incubated with increasing amounts EGF-¹¹¹In-labeled transferrin composition or ¹¹¹In-oxine, centrifuged to remove free ligand, assayed and then seeded into 50 mm culture dishes. The number of cells seeded was varied from 3×10⁴ to 3×10⁶cells to obtain approximately 400 viable colonies/dish taking into account the plating efficiency and the expected level of cytotoxicity. Control dishes contained JJ5 breast cancer cells which were incubated with normal saline. Growth medium was added and the cells were cultured at 37° C./5% CO² for 14 days. The growth medium was removed and the colonies were stained with methylene blue (1% in a 1:1 mixture of ethanol and water) then washed twice. The number of colonies per dish was counted using a manual colony counter (Manostat Corp). The plating efficiency was calculated by dividing the number of colonies observed by the number of cells seeded in each dish. The surviving fraction at increasing amounts of EGF-¹¹¹In-labeled transferrin composition or ¹¹¹In-oxine was calculated by dividing the plating efficiency for dishes containing treated cells with that observed for control dishes with normal saline.

Using a colony-forming assay, the radiotoxicity of internalization for JJ5 breast cancer cells was evaluated. EGF-¹¹¹In-labeled transferrin compositions(8 pCi/cell) resulted in a 99% reduction in cell survival for JJ5 cells. ¹¹¹In-oxine was also radiotoxic with greater than 99% cell killing at <6 pCi/cell.

There are various advantages of using the methods of the instant invention in cancer therapy. As seen from the foregoing data, EGF-¹¹¹In-labeled transferrin compositions are rapidly internalized by cancer cells. The internalization process for EGF-¹¹¹In-labeled transferrin compositions involves an active transport mechanism utilizing the EGFR binding domain of the composition, rather than simple diffusion across the cell membrane. This active transport mechanism for the composition probably also includes nuclear translocation, as for the case of EGF, which allows for a maximal radiation dose of Auger electrons to be delivered to the cell's DNA. The compositions used in the methods of the instant invention employ human polypeptides and are not immunogenic in humans. EGF-¹¹¹In-labeled transferrin compositions have been shown to retain ¹¹¹In over a 24 hour period at 37° C., with <8% of ¹¹¹In radioactivity was lost from cells in vitro. These characteristics are important for cell killing.

Immunohistochemistry Staining and Measurement of EGF Receptor on BCBM Cells

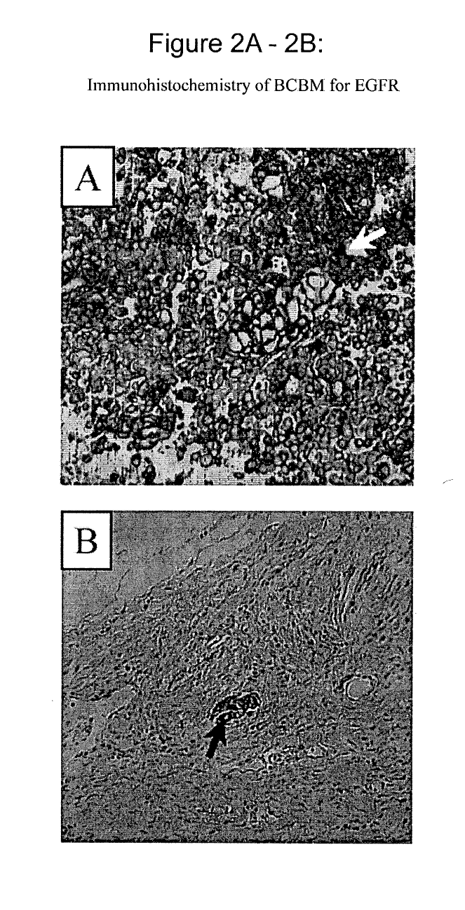

Immunohistochemistry of BCBM pre-implanted into mice showed all the specimens (n=20) had breast cancer cells negative for the estrogen and progesterone receptors (data not shown). Normal human bone histological sections were used as controls, no staining was observed in these specimens (data not shown). BCBM were retrieved from the mice at 20 weeks. Histologic sections were fixed and prepared. Immunohistochemical staining was done using mouse anti-EGF-receptor monoclonal antibody (TS40). In contrast to the implants and the controls, 16 of the 20 BCBM specimens had breast cancer cells positive for human EGFR (see FIGS. 2A-B). The white arrow in FIG. 2A points out a dense mass of EGFR+ cells. The arrow in FIG. 2B points out an isolated EGFR+ cell in the bone marrow. Mean (±SDEV) expression levels of EGF receptor was measured on breast cancer cells from tumor JJ5 by radioligand binding assay 24 and were in the range of 2.7 (±0.8)×10⁶ receptors/cell.

Immunohistochemistry Staining of BCBM Human Blood Vessels

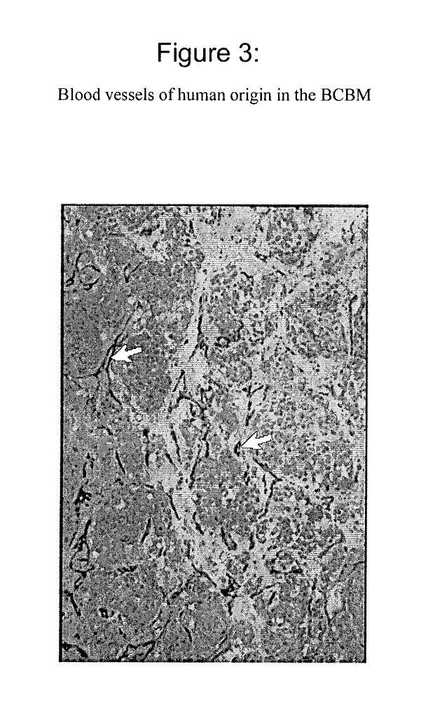

To evaluate the role of angiogenesis in the growth of human breast carcinoma, human BCBM surgical specimens were implanted in SCID mice. The breast tumors showed numerous blood vessels infiltrating the central mass of the tumors. In order to accurately assess the efficacy of treatment using the methods of the instant invention against human tumors, the blood vessels which developed in the BCBM in the mice must be of human origin. Immunohistochemical staining was done on BCBM sections using mouse anti-human CD34 antibody. Anti-human CD34 reacts specifically with human blood vessels and thus will not react with murine blood vessels. As shown in FIG. 3, these results clearly demonstrate the presence of human blood vessel angiogenesis within the tumor xenografts retrieved from SCID mice at 20 weeks. In FIG. 3, the arrow points out the dark blood vessels of human origin (stained with anti-human CD34), thus these specimens can be used to accurately assess the efficacy of the VEGF portion of the composition used in the methods of the instant invention.

Cell Culture Studies Using Composition Containing Doxorubicin

JJ5 breast cancer cells expressing approximately 10⁶ epidermal growth factor receptors/cell were incubated with EGF-NHS-PEG3400-Biotin-TF-SA-Doxorubicin [EGF-Transferrin-Doxorubicin], or EGF-NHS-PEG3400-Biotin-TF-SA or Doxorubicin, centrifuged to remove test material, then and seeded (10⁶ cells/dish) into 35 mm culture dishes. Growth medium was added and the cells were cultured at 37° C./5% CO² for 4 days, the cells were then recovered by trypsinization and counted in a hemocytometer. Control dishes contained cells cultured in growth medium containing EGF-NHS-PEG3400-Biotin-TF-SA. Cytotoxicity Assay of JJ5 breast cancer cells expressing approximately 10⁶ epidermal growth factor receptors/cell were incubated with increasing amounts EGF-NHS-PEG3400-Biotin-TF-SA-Doxorubicin or Doxorubicin, centrifuged to remove free test material, assayed and then seeded into 50 mm culture dishes. The number of cells seeded was varied from 3×10⁴ to 3×10⁶ cells to obtain approximately 350 viable colonies/dish taking into account the plating efficiency and the expected level of cytotoxicity. Control dishes contained JJ5 breast cancer cells which were incubated with normal saline. Growth medium was added and the cells were cultured at 37° C./5% CO2 for 4 days. The growth medium was removed and the colonies were stained with methylene blue (1% in a 1:1 mixture of ethanol and water) then washed twice. The number of colonies per dish was counted using a manual colony counter (Manostat Corp). The plating efficiency was calculated by dividing the number of colonies observed by the number of cells seeded in each dish. The surviving fraction at increasing amounts of EGF-NHS-PEG3400-Biotin-TF-SA-Doxorubicin or Doxorubicin was calculated by dividing the plating efficiency for dishes containing treated cells with that observed for control dishes with normal saline. The growth inhibition assay of EGF-NHS-PEG3400-Biotin-TF-SA-Doxorubicin achieved 86% growth inhibition of the JJ5 cells compared to the medium control whereas Doxorubicin which enters all the cells resulted in 95% growth inhibition.

Animal Studies

Effect of Compositions Containing Doxorubicin on BCBM Growth

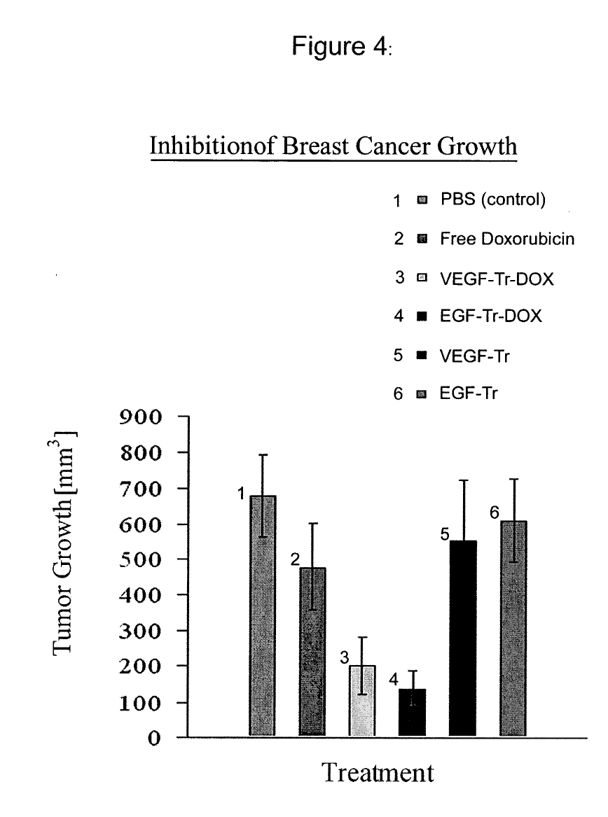

SCID mice were implanted with BCBM, JJ5. Control (Group 1) BCBM-SCID mice were treated intraperitoneally with PBS solution once a week for 5 weeks. Experimental group 2 BCBM-SCID mice were treated intraperitoneally with free doxorubicin (100 ug) once a week for 5 weeks. Experimental group 3 BCBM-SCID mice were treated intraperitoneally with VEGF-transferrin-doxorubicin (100 ug) once a week for 5 weeks. Experimental group 4 BCBM-SCID mice were treated intraperitoneally with EGF-transferrin-doxorubicin (100 ug) once a week for 5 weeks. Experimental group 5 BCBM-SCID mice were treated intraperitoneally with VEGF-transferrin (100 ug) once a week for 5 weeks. Experimental group 6 BCBM-SCID mice were treated intraperitoneally with EGF-transferrin (100 ug) once a week for 5 weeks. At the end of the experiment the BCBM were resected from both control and experimental mice and tumor weight and volume were determined. Maximum inhibition of tumor growth is obtained by treatment with EGF-transferrin-doxorubicin. The results of this experiment are illustrated in the bar graph of FIG. 4. In the bar graph presented by FIG. 4, bar #1 represents the tumor volume seen in control mice treated with PBS solution, bar #2 represents the tumor volume seen in mice administered free doxorubicin, bar #3 represents the tumor volume seen in mice administered VEGF-transferrin-doxorubicin, bar #4 represents the tumor volume seen in mice administered EGF-transferrin-doxorubicin, bar #5 represents the tumor volume seen in mice administered VEGF-transferrin and bar #6 represents the tumor volume seen in mice administered EGF-transferrin. The P values representing the statistical significance of inhibition of tumor growth as compared with tumor growth of the control are as follows: bar #2 0.266257; bar #3 0.099692; bar #4 0.006155; bar #5 0.550571 and bar #6 0.6786. A maximum, statistically significant amount of inhibition of tumor growth (BCBM) was seen in mice treated with EGF-transferrin-doxorubicin shown by bar #4 in FIG. 4.

In summary, the methods of the instant invention enable selective delivery of anti-tumor agents to a host having a tumor. As is evidenced by the experimental examples described and shown herein, the instant invention provides a therapeutic method involving a composition capable of selectively concentrating anti-tumor agents to the tumor cells and associated tumor vasculature while simultaneously reducing the concentration of anti-tumor agents in non-diseased tissues.

All patents and publications mentioned in this specification are indicative of the levels of those skilled in the art to which the instant invention pertains. All patents and publications are herein incorporated by reference to the same extent as if each individual patent and publication was specifically and individually indicated to be incorporated by reference.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification.

One skilled in the art will readily appreciate that the present invention is well adapted to carry out the objects and obtain the ends and advantages mentioned, as well as those inherent therein. The oligonucleotides, peptides, polypeptides, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed various modifications of the described modes for carrying out the invention which are obvious to those skilled in the art are intended to be within the scope of the following claims.

What is claimed is:

1. A method for inhibiting the growth of tumor tissue, said method comprising the steps of:
(a) administering a biologically effective amount of a compound to a host having a tumor, said compound comprising human vascular endothelial growth factor (VEGF) and at least one anti-tumor agent each operatively linked to human transferrin, and
(b) repeating said administering of step (a) over a period of time until a statistically significant inhibition of tumor growth is achieved.

2. The method in accordance with claim 1 wherein said at least one anti-tumor agent is selected from the group comprising doxorubicin, daunorubicin, idarubicin, mitoxantrone, bleomycin, dactinomycin, carminomycin, detorubicin, epirubicin, esorubicin, mitomycin C, plicamycin and streptozocin.

3. The method in accordance with claim 1 wherein said at least one anti-tumor agent is doxorubicin.

4. A method for inhibiting the growth of tumor tissue, said method comprising the steps of:
(a) administering a biologically effective amount of a compound to a host having a tumor, said compound comprising human vascular endothelial growth factor (VEGF) and at least one anti-tumor agent each operatively linked to radiolabeled human transferrin, and
(b) repeating said administering of step (a) over a period of time until a statistically significant inhibition of tumor growth is achieved.

5. The method in accordance with claim 4 wherein the radiolabel on said radiolabeled human transferrin is selected from the group comprising ¹¹¹In, ⁶⁷GA and ⁶⁸Ga.

6. The method in accordance with claim 4 wherein the radiolabel on said radiolabeled human transferrin comprises ¹¹¹In.

7. The method in accordance with claim 4 wherein said at least one anti-tumor agent is selected from the group comprising doxorubicin, daunorubicin, idarubicin, mitoxantrone, bleomycin, dactinomycin, carminomycin, detorubicin, epirubicin, esorubicin, mitomycin C, plicamycin and streptozocin.

8. The method in accordance with claim 5 wherein said at least one anti-tumor agent is selected from the group comprising doxorubicin, daunorubicin, idarubicin, mitoxantrone, bleomycin, dactinomycin, carminomycin, detorubicin, epirubicin, esorubicin, mitomycin C, plicamycin and streptozocin.

9. The method in accordance with claim 6 wherein said at least one anti-tumor agent is selected from the group comprising doxorubicin, daunorubicin, idarubicin, mitoxantrone, bleomycin, dactinomycin, carminomycin, detorubicin, epirubicin, esorubicin, mitomycin C, plicamycin and streptozocin.

10. The method in accordance with claim 4 wherein said at least one anti-tumor agent is doxorubicin.

11. The method in accordance with claim 5 wherein said at least one anti-tumor agent is doxorubicin.

12. The method in accordance with claim 6 wherein said at least one anti-tumor agent is doxorubicin.

13. A method for inhibiting the growth of tumor tissue, said method comprising the steps of:
(a) administering a biologically effective amount of a conjugate to a host having a tumor, said conjugate consisting essentially of human vascular endothelial growth factor (VEGF)and at least one anti-tumor agent each operatively linked to human transferrin, and
(b) repeating said administering of step (a) over a period of time until a statistically significant inhibition of tumor growth is achieved.

14. The method in accordance with claim 13 wherein said at least one anti-tumor agent is selected from the group comprising doxorubicin, daunorubicin, idarubicin, mitoxantrone, bleomycin, dactinomycin, carminomycin, detorubicin, epirubicin, esorubicin, mitomycin C, plicamycin and streptozocin.

15. The method in accordance with claim 13 wherein said at least one anti-tumor agent is doxorubicin.

16. A method for inhibiting the growth of tumor tissue, said method comprising the steps of:
(a) administering a biologically effective amount of a conjugate to a host having a tumor, said conjugate consisting essentially of human vascular endothelial growth factor (VEGF) and at least one anti-tumor agent each operatively linked to radiolabeled human transferrin, and
(b) repeating said administering of step (a) over a period of time until a statistically significant inhibition of tumor growth is achieved.

17. The method in accordance with claim 16 wherein the radiolabel on said radiolabeled human transferrin is selected from the group comprising ¹¹¹In, ⁶⁷GA and ⁶⁸Ga.

18. The method in accordance with claim 16 wherein the radiolabel on said radiolabeled human transferrin comprises ¹¹¹In.

19. The method in accordance with claim 16 wherein said at least one anti-tumor agent is selected from the group comprising doxorubicin, daunorubicin, idarubicin, mitoxantrone, bleomycin, dactinomycin, carminomycin, detorubicin, epirubicin, esorubicin, mitomycin C, plicamycin and streptozocin.

20. The method in accordance with claim 17 wherein said at least one anti-tumor agent is selected from the group comprising doxorubicin, daunorubicin, idarubicin, mitoxantrone, bleomycin, dactinomycin, carminomycin, detorubicin, epirubicin, esorubicin, mitomycin C, plicamycin and streptozocin.

21. The method in accordance with claim 18 wherein said at least one anti-tumor agent is selected from the group comprising doxorubicin, daunorubicin, idarubicin, mitoxantrone, bleomycin, dactinomycin, carminomycin, detorubicin, epirubicin, esorubicin, mitomycin C, plicamycin and streptozocin.

22. The method in accordance with claim 16 wherein said at least one anti-tumor agent is doxorubicin.

23. The method in accordance with claim 17 wherein said at least one anti-tumor agent is doxorubicin.

24. The method in accordance with claim 18 wherein said at least one anti-tumor agent is doxorubicin.

25. A method for inhibiting the growth of tumor tissue, said method comprising the steps of:
(a) administering a biologically effective amount of a compound to a host having a tumor, said compound comprising human epidermal growth factor (EGF) and at least one anti-tumor agent each operatively linked to human transferrin, and
(b) repeating said administering of step (a) over a period of time until a statistically significant inhibition of tumor growth is achieved.

26. The method in accordance with claim 25 wherein said at least one anti-tumor agent is selected from the group comprising doxorubicin, daunorubicin, idarubicin, mitoxantrone, bleomycin, dactinomycin, carminomycin, detorubicin, epirubicin, esorubicin, mitomycin C, plicamycin and streptozocin.

27. The method in accordance with claim 25 wherein said at least one anti-tumor agent is doxorubicin.

28. A method for inhibiting the growth of tumor tissue, said method comprising the steps of:
(a) administering a biologically effective amount of a compound to a host having a tumor, said compound comprising human epidermal growth factor (EGF) and at least one anti-tumor agent each operatively linked to radiolabeled human transferrin, and
(b) repeating said administering of step (a) over a period of time until a statistically significant inhibition of tumor growth is achieved.

29. The method in accordance with claim 28 wherein the radiolabel on said radiolabeled human transferrin is selected from the group comprising ¹¹¹In, ⁶⁷GA and ⁶⁸Ga.

30. The method in accordance with claim 28 wherein the radiolabel on said radiolabeled human transferrin comprises ¹¹¹In.

31. The method in accordance with claim 28 wherein said at least one anti-tumor agent is selected from the group comprising doxorubicin, daunorubicin, idarubicin, mitoxantrone, bleomycin, dactinomycin, carminomycin, detorubicin, epirubicin, esorubicin, mitomycin C, plicamycin and streptozocin.

32. The method in accordance with claim 29 wherein said at least one anti-tumor agent is selected from the group comprising doxorubicin, daunorubicin, idarubicin, mitoxantrone, bleomycin, dactinomycin, carminomycin, detorubicin, epirubicin, esorubicin, mitomycin C, plicamycin and streptozocin.

33. The method in accordance with claim 30 wherein said at least one anti-tumor agent is selected from the group comprising doxorubicin, daunorubicin, idarubicin, mitoxantrone, bleomycin, dactinomycin, carminomycin, detorubicin, epirubicin, esorubicin, mitomycin C, plicamycin and streptozocin.

34. The method in accordance with claim 28 wherein said at least one anti-tumor agent is doxorubicin.

35. The method in accordance with claim 29 wherein said at least one anti-tumor agent is doxorubicin.

36. The method in accordance with claim 30 wherein said at least one anti-tumor agent is doxorubicin.

37. A method for inhibiting the growth of tumor tissue, said method comprising the steps of:
(a) administering a biologically effective amount of a conjugate to a host having a tumor, said conjugate consisting essentially of human epidermal growth factor (EGF)and at least one anti-tumor agent each operatively linked to human transferrin, and
(b) repeating said administering of step (a) over a period of time until a statistically significant inhibition of tumor growth is achieved.

38. The method in accordance with claim 37 wherein said at least one anti-tumor agent is selected from the group comprising doxorubicin, daunorubicin, idarubicin, mitoxantrone, bleomycin, dactinomycin, carminomycin, detorubicin, epirubicin, esorubicin, mitomycin C, plicamycin and streptozocin.

39. The method in accordance with claim 37 wherein said at least one anti-tumor agent is doxorubicin.

40. A method for inhibiting the growth of tumor tissue, said method comprising the steps of:
(a) administering a biologically effective amount of a conjugate to a host having a tumor, said conjugate consisting essentially of human epidermal growth factor (EGF) and at least one anti-tumor agent each operatively linked to radiolabeled human transferring and
(b) repeating said administering of step (a) over a period of time until a statistically significant inhibition of tumor growth is achieved.

41. The method in accordance with claim 40 wherein the radiolabel on said radiolabeled human transferrin is selected from the group comprising ¹¹¹In, ⁶⁷GA and ⁶⁸Ga.

42. The method in accordance with claim 40 wherein the radiolabel on said radiolabeled human transferrin comprises ¹¹¹In.

43. The method in accordance with claim 40 wherein said at least one anti-tumor agent is selected from the group comprising doxorubicin, daunorubicin, idarubicin, mitoxantrone, bleomycin, dactinomycin, carminomycin, detorubicin, epirubicin, esorubicin, mitomycin C, plicamycin and streptozocin.

44. The method in accordance with claim 41 wherein said at least one anti-tumor agent is selected from the group comprising doxorubicin, daunorubicin, idarubicin, mitoxantrone, bleomycin, dactinomycin, carminomycin, detorubicin, epirubicin, esorubicin, mitomycin C, plicamycin and streptozocin.

45. The method in accordance with claim 42 wherein said at least one anti-tumor agent is selected from the group comprising doxorubicin, daunorubicin, idarubicin, mitoxantrone, bleomycin, dactinomycin, carminomycin, detorubicin, epirubicin, esorubicin, mitomycin C, plicamycin and streptozocin.

46. The method in accordance with claim 40 wherein said at least one anti-tumor agent is doxorubicin.

47. The method in accordance with claim 41 wherein said at least one anti-tumor agent is doxorubicin.

48. The method in accordance with claim 42 wherein said at least one anti-tumor agent is doxorubicin.


SEQUENCE LISTING (8 sequences)

SEQ ID NO: 1 - DNA, 495 bp, Homo sapiens
gcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcgg

SEQ ID NO: 2 - protein, 165 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 3 - DNA, 2037 bp, Homo sapiens
gtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacttggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctagacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtccccccaaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagacct

SEQ ID NO: 4 - protein, 679 aa, Homo sapiens
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 5 - DNA, 159 bp, Homo sapiens
aactctgattccgaatgcccgctgtctcatgacggttactgcctgcatgatggcgtatgcatgtacatcgaagctctggacaaatacgcatgcaactgtgttgtaggttacatcggcgaacgttgccagtatcgcgacctgaaatggtgggaactgcgt

SEQ ID NO: 6 - protein, 53 aa, Homo sapiens
NSDSECPLSHDGYCLHDGVCMYIEALDKYACNCVVGYIGERCQYRDLKWWELR

SEQ ID NO: 7 - DNA, 45 bp, Artificial
  sequence codes for a polylinker
ggtggcggtggctcgggcggtggtgggtcgggtggcggcggatct

SEQ ID NO: 8 - protein, 15 aa, Artificial
  sequence of a polylinker
GGGGSGGGGSGGGGS